(19) United States
(12) Patent Application Publication
(10) Pub. No.: US 2010/0286445 A1
(43) Pub. Date: Nov. 11, 2010

(54) METHOD OF FINISHING ORGANIC PIGMENTS

(75) Inventors: Matthias Klueglein, Ludwigshafen (DE); Richard Van Gelder, Speyer (DE); Joachim Jesse, Weisenheim (DE); Wolfgang Best, Freinsheim (DE); Benno Sens, Neustadt (DE); Rainer Henning, Freinsheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/671,829

(22) PCT Filed: Jul. 30, 2008
(86) PCT No.: PCT/EP08/59996

§ 371 Date: Feb. 2, 2010

(30) Foreign Application Priority Data
Aug. 3, 2007 (EP) 07113797.0

(51) Int. Cl. C07C 233/76

(52) U.S. Cl. 564/180

(57) ABSTRACT

A method of finishing an organic pigment that involves dissolving or dispersing the pigment in a mineral acid and crystallizing the pigment from the solution or dispersion by mixing with an aqueous diluent in the absence of a sulfonato-functional condensation product of an arylsulfonic acid and an aliphatic aldehyde as crystallization inhibitor, which comprises ripening the crystallized organic pigment in the presence of a surfactant or in the presence of a pigment solubility enhancer in aqueous suspension.

### DESCRIPTION

The invention relates to a method of finishing organic pigments.

Organic pigments are frequently obtained from their synthesis in a coarsely crystalline form with a very heterogeneous particle size distribution. For conversion into a coloristically valuable pigment form appropriate to the application, therefore, the crude pigments are typically subjected to a finishing operation.

Known for these purposes is the grinding of the crude pigment and subsequent recrystallization of the ground material from an organic solvent, or the grinding is carried out as wet grinding in aqueous suspension in high-speed agitated ball mills. Although the methods described do achieve—in some cases in a very time-consuming way—a comminution of the particles and hence an improvement in the coloristic properties of the pigments, the particle size of the pigments obtained is difficult to control, and the pigments frequently have a particle size distribution which is inadequate—being too broad—for a range of applications: for example, as colorants for metallic paint for the automobile industry.

WO 02/00643 discloses a method of finishing crude quinophthalone pigments by subjecting the as-synthesized crude pigment to grinding in the absence of grinding assistants and subsequently crystallizing the resulting ground material from an organic solvent or from a mixture of organic solvent and water in the presence of a quinophthalone derivative. Derivatives specified include, for example, sulfonic acid derivatives of the quinophthalone pigments.

WO 2004/048482 discloses a method of finishing organic pigments by dissolving the pigment in concentrated sulfuric acid and mixing the sulfuric acid solution with water in the presence of a condensation product of naphthalenesulfonic acid and formaldehyde as a crystallization modifier. This crystallization modifier is added prior to the mixing of the sulfuric-acid pigment solution, or is generated in situ therein by reaction of 1- and 2-naphthalenesulfonic acid with formaldehyde. A disadvantage of this in situ synthesis is that it results in condensation products with a broad and poorly defined molecular weight distribution.

It is an object of the invention to provide an advantageous and easy-to-implement method of finishing organic pigments that produces pigments having very good coloristic properties.

This object is achieved by a method of finishing an organic pigment that involves dissolving or dispersing the pigment in a mineral acid and crystallizing the pigment from the solution or dispersion by mixing with an aqueous diluent in the absence of a sulfonato-functional condensation product of an arylsulfonic acid and an aliphatic aldehyde, which comprises ripening the crystallized organic pigment in the presence of a surfactant or in the presence of a pigment solubility enhancer in aqueous suspension.

The step of crystallization by mixing of the sulfuric-acid pigment solution with the aqueous diluent is carried out explicitly in the absence of a condensation product, acting as a crystallization inhibitor, of an arylsulfonic acid and an aliphatic aldehyde. The polymeric crystallization inhibitors are typically condensation products of one or more naphthalenesulfonic acids such as 1-naphthalenesulfonic acid, 2-naphthalene-sulfonic acid or mixtures thereof and one or more different aliphatic aldehydes, typically formaldehyde. Additionally, condensation products of one or more different hydroxy-arylsulfonic acids and an aliphatic aldehyde and also, if appropriate, urea and, if appropriate, alkali metal sulfite are used as such crystallization inhibitors.

In spite of foregoing the presence of a polymeric crystallization inhibitor during the precipitation of the pigment from its sulfuric-acid solution, the method of the invention allows pigments to be obtained that have very good coloristic properties and performance properties. At the same time a method is realized which, overall, is highly economic.

In the finishing method of the invention the organic pigment is dissolved or dispersed in a mineral acid and is crystallized from the solution or dispersion by dilution with an aqueous diluent in the absence of a polymeric crystallization inhibitor. A preferred mineral acid is sulfuric acid. More particularly the pigment is dissolved in concentrated sulfuric acid. In this case the surfactant or pigment solubility enhancer is already present in the aqueous diluent when the mineral-acid pigment solution is mixed with the aqueous diluent, or it is added subsequently. It may also be present in the mineral-acid pigment solution or in the diluent and the pigment solution. If may also be added only to a pigment which has first been isolated as a solid and then redispersed. In one preferred variant of this method, mixing is accomplished by combining the mineral-acid pigment solution and the aqueous diluent using a mixing nozzle. The aqueous diluent is generally water. Crystallization may also take place by the pouring of the mineral-acid pigment solution into the aqueous diluent—preferably ice-water.

Generally speaking, the concentration of the pigment in the mineral-acid pigment solution is 1% to 30% by weight. The volume of the admixed aqueous diluent is generally 1 to 12 times the volume of the mineral-acid pigment solution.

In one embodiment, following crystallization, the crystallized organic pigment, present in the form of its aqueous suspension in the aqueous diluent, can be ripened in the presence of a surfactant. The surfactant may be added to the aqueous diluent itself or may be added after the step of crystallization of the pigment suspension, addition taking place generally in the form of an aqueous solution. The surfactant may also be added with the mineral-acid pigment solution. The surfactant is used generally in amounts of 0.1% to 30% by weight, based on the pigment. The ripening of the crystallized organic pigment takes place in general by stirring of the pigment suspension at temperatures of generally 40 to 150° C. over a period of 0.5 to 5 h. In the course of this time, larger pigment particles grow at the expense of smaller pigment particles and/or there is smoothing/healing of the crystal surface of the pigment particles. Since operation takes place in the presence of a surfactant, this procedure occurs as early as in the aqueous sulfuric-acid pigment suspension, with comparatively low sulfuric acid content, of the kind that is present after the sulfuric-acid pigment solution has been mixed with the aqueous diluent.

Suitable surfactants are the anionic, cationic, nonionic, and amphoteric surfactants specified below.

Examples of suitable surfactants are anionic surfactants such as alkylbenzene-sulfonates or alkylnaphthalenesulfonates or alkylsulfosuccinates, cationic surfactants such as quaternary ammonium salts, benzyltributylammonium chloride for example, or nonionic or amphoteric surfactants such as polyoxyethylene surfactants and alkyl or amidopropyl betaines. The surfactants may be present during the precipitation step or may not be added until later.

Preferred surfactants are nonionic surfactants, examples being alkoxylates of hydroxyaromatics, more particularly alpha- or beta-naphthol alkoxylates having 2 to 40 oxyalkylene units, such as alpha- or beta-naphthol ethoxylates having 2 to 40 oxyethylene units.

In one embodiment, following crystallization, the crystallized organic pigment, which is in the form of its aqueous suspension in the aqueous diluent, can be ripened in the presence of a pigment solubility enhancer. The solubility enhancer added is generally an organic solvent. Suitable organic solvents are, for example, xylenes, glycols, alcohols, THF, acetone, NMP, DMF, and nitrobenzene. These solvents are added generally in an amount of 0.1% to 50% by weight, based on the aqueous pigment suspension. The optimum amount of solvent can be determined by means of rangefinding tests for each solvent. Generally speaking, the suspension is stirred, in the presence of the organic solvent, at temperatures in the range from 15° C. to boiling temperature over a period of 30 min to 5 h, after which the organic solvent is removed by distillation.

In a further embodiment of the method of the invention the pigment obtained by precipitation is first isolated as a solid and then dispersed in water or in an aqueous solvent mixture, which may already contain the surfactant or the pigment solubility enhancer, and the dispersed pigment is ripened in the presence of the surfactant and/or the solubility enhancer. The surfactant or the pigment solubility enhancer may also be added afterward to the aqueous suspension of the pigment.

The surfactant and the solubility enhancer act generally as crystallization modifiers. In the course of ripening, larger pigment particles grow at the expense of smaller pigment particles and/or there is smoothing/healing of the crystal surfaces of the pigment particles.

In all cases the crystallized and finished pigment is subsequently isolated in solid form by filtration of the aqueous suspension.

Examples of suitable pigments that can be finished by the method of the invention are azo, azomethine, methine, anthraquinone, phthalocyanine, perinone, perylene, diketopyrrolopyrrole, thioindigo, thiazineindigo, dioxazine, iminoisoindoline, iminoisoindolinone, quinacridone, flavanthrone, indanthrone, anthrapyrimidine and quinophthalone pigments. Preferred pigments are phthalocyanine, perylene, quinaeridone, indanthrone, and quinophthalone pigments, dioxazine and diketopyrrolo-pyrrole; particularly preferred pigments are phthalocyanine, perylene, and indanthrone pigments.

Preferred among the perylenes are the pigments of the C.I. Pigment Red 179 type. These pigments can be prepared by a variety of processes. For instance, C.I. Pigment Red 179 can be prepared by methylating perylene-3,4,9,10-tetracarboxylic diimide with a methylating agent or by condensing perylenetetracarboxylic anhydride with methylamine. The latter process is used with preference. An alternative is to use mixtures of the pigments prepared by these two methods. It is also possible to use pigment derivatives prepared by these methods, or mixtures of said derivatives.

In certain cases it can be of advantage in addition to use further crystallization modifiers such as specific polymers or dispersants which act as crystallization inhibitors. Examples of such dispersants are sulfonamides or pigment derivatives comprising sulfonic acid groups, such as imidazolemethyl- or pyrazolemethyl-quinacridone pigment sulfonic acids. These further crystallization modifiers may be present during the precipitation step or may not be added until later. Examples of suitable specific polymers are polyacrylic acid, polymethacrylic acid, polyurethanes, polyvinyl alcohol, polyvinylpyrrolidone or cellulose derivatives. They may be present during the precipitation step or not added until later.

It is possible for further finishing steps to follow. For instance, preferably, the pigment isolated in solid form is blended with a pigment synergist. This synergist is generally a derivative comprising sulfonate groups or carbonate groups, or a basic derivative, of the aforementioned pigments. The pigment synergist is preferably the sulfonato-functional derivative of the finished pigment with which the synergist is blended. Generally speaking, the pigment synergist is used in amounts of 0.1% to 15% by weight, preferably 0.5% to 10% by weight, based on the completed pigment formulation.

The average particle size of the finished pigments is generally in the range from 10 to 400 nm, preferably 20 to 200 nm.

The pigments finished by the method of the invention may comprise the crystallization modifier, i.e., the surfactant or the further crystallization modifiers or crystallization inhibitors, on the surface of the pigment particles. In addition to the pigment synergists already specified, the pigment preparations may comprise further additives, generally in amounts of up to 15% by weight. Examples of further additives are wetting agents, antifoams, antioxidants, UV absorbers, stabilizers, plasticizers, and texturing auxiliaries.

The invention is illustrated by the examples below.

### EXAMPLES

### Comparative Example

A solution of 80 g of C.I. P.B. 60 in 987 g of 96% strength sulfuric acid is stirred for two hours, during which it is kept at a temperature of 25° C. This solution is combined via a mixing nozzle with 2.5 parts of water. The temperature at this stage climbs to 60° C. The system is subsequently stirred at 60° C. for 30 minutes. The suspension is filtered and the filter product is washed with deionized water and then dried under vacuum in a drying oven at 80° C. It is then ground in a coffee mill at maximum speed for 20 seconds. The resulting pigment combines a high dispersion hardness with poor flow behavior in the application matrix: for example, a paint formulation.

### Example 1

1 part of a pigment solution, prepared as in the Comparative Example, is combined via a mixing nozzle with 2.5 parts of water. At this stage the temperature climbs to 60° C. The system is subsequently stirred at 60° C. for 30 minutes. The suspension is filtered and the filtercake is washed with deionized water. Subsequently the filtercake is stirred again in deionized water, and the dispersion is introduced into a jacketed reactor and heated to 60° C. over the course of one hour. At this temperature 20 g of p-xylene are added and the system is stirred for two hours. Subsequently xylene is removed by distillation at 100° C. The suspension is then filtered and the filtercake is washed with a little water and dried in a vacuum drying oven at 80° C. The pigment combines a low dispersion harshness with good flow behavior and high transparency.

### Example 2

1 part of a pigment solution, prepared as in the Comparative Example, is combined via a mixing nozzle with 2.5 parts of water. At this stage the temperature climbs to 60° C. The system is subsequently stirred at 60° C. for 30 minutes. The suspension is filtered and the filtercake is washed with deionized water. Subsequently the filtercake is stirred again in 480 parts of deionized water. 6 parts of a beta-naphthol ethoxylate (Lugalvan BNO 12, BASF AG, Ludwigshafen, DE) are added and the batch is heated to 85° C. and stirred at 85° C. for 5 hours. After that the batch is filtered and the filter product is washed to neutrality with deionized water and dried in a forced-air oven at 75° C. The pigment combines a low dispersion harshness with good flow behavior and high transparency.

### Example 3

The procedure of Example 2 is repeated. Subsequently 95 parts of the pigment are ground together with 5 parts of Solsperse 12000 (Noveon). The aqueous paint system comprising this pigment has an even lower viscosity than that with the pigment from Example 2.

### Example 4

1 part of a pigment solution, prepared as in the Comparative Example, is combined via a mixing nozzle with 2.5 parts of water. At this stage the temperature climbs to 60° C. The system is subsequently stirred at 60° C. for 30 minutes. 6 parts of a beta-naphthol ethoxylate (Lugalvan BNO 12, BASF AG, Ludwigshafen, DE) are added and the batch is heated to 85° C. and stirred at 85° C. for 5 hours. After that the batch is filtered and the filter product is washed to neutrality with deionized water and dried in a forced-air oven at 75° C. The pigment combines a low dispersion harshness with good flow behavior and high transparency. The quality of the pigment is the same as that of the pigment from Example 2.

1. A method of finishing an organic pigment that involves dissolving or dispersing the pigment in a mineral acid and crystallizing the pigment from the solution or dispersion by mixing with an aqueous diluent in the absence of a sulfonato-functional condensation product of an arylsulfonic acid and an aliphatic aldehyde as crystallization inhibitor, said method comprising ripening the crystallized organic pigment in the presence of a surfactant or in the presence of a pigment solubility enhancer in aqueous suspension.

2. The method according to claim 1, wherein the mixing with the aqueous diluent takes place by means of a mixing nozzle.

3. The method according to claim 1, wherein the surfactant or the solubility enhancer is present during mixing in the aqueous diluent or is not added until after the crystallization step.

4. The method according to claim 1, wherein the crystallized pigment is isolated as a solid, redispersed in water or an aqueous solvent mixture, and ripened in the presence of the surfactant or of the pigment solubility enhancer.

5. The method according to claim 1, wherein the pigment sollubility enhancer is selected from xylenes, glycols, alcohols, THF, acetone, NMP, DMF, and nitrobenzene.

6. The method according to claim 1, wherein the surfactant is selected from anionic, cationic, nonionic or amphoteric surfactants.

7. The method according to claim 6, wherein the surfactant is a nonionic surfactant.

8. The method according to claim 7, wherein the surfactant is an alpha- or beta-naphthol alkoxylate.

9. The method according to claim 1, wherein, in a further step, the pigment isolated in solid form is blended with a pigment synergist which is a derivative comprising sulfonate groups or carbonate groups or is a basic derivative of an organic pigment.

10. The method according to claim 9, wherein the pigment synergist is a derivative of the finished pigment.

11. The method according to claim 1, wherein the pigment is selected from the group consisting of azo, azomethine, methine, anthraquinone, phthalocyanine, perinone, perylene, diketopyrrolopyrrole, thioindigo, thiazineindigo, dioxazine, iminoisoindoline, iminoisoindolinone, quinacridone, flavanthrone, indanthrone, anthrapyrimidine, and quinophthalone pigments.
